(19) United States
(12) Patent Application Publication
(10) Pub. No.: US 2003/0229101 A1
(43) Pub. Date: Dec. 11, 2003

(54) Tablets comprising ciprofloxacin hydrochloride

(76) Inventor: Bernard Charles Sherman, Toronto (CA)

Correspondence Address: NIXON & VANDERHYE P.C., 1100 North Glebe Rd., 8th Floor, Arlington, VA, 22201-4714, US

(21) Appl. No.: 10/163,900

(22) Filed: Jun. 6, 2002

(51) Int. Cl. A61K 31/496; A61K 9/20

(52) U.S. Cl. 514/253.08; 424/465

(57) ABSTRACT

A pharmaceutical tablet comprising over 80% by weight ciprofloxacin hydrochloride monohydrate.

### BACKGROUND OF THE INVENTION

Ciprofloxacin is a broad spectrum antimicrobial agent sold in the United States and elsewhere under the tradename Cipro™. Cipro™ tablets comprise ciprofloxacin (as the hydrochloride monohydrate) in strengths of 100 mg, 250 mg, 500 mg and 750 mg. Since the molecular weight of the ciprofloxacin hydrochloride monohydrate is 385.82 daltons, versus 331.34 daltons for ciprofloxacin, it follows that the 750 mg tablets, for example, must contain 750×385.81/331.34 mg or 873 mg of ciprofloxacin hydrochloride monohydrate per tablet. Since the tablets must also contain excipients (i.e. inactive ingredients), a Cipro™ tablet of 750 mg strength is relatively large. ™—Registered trademark

Cipro™ 750 mg tablets weigh about 1150 mg each. They are capsule-shaped with a length of about ⅞ inch, and are relatively difficult to swallow because of the large size. It would thus be desirable to enable tablets smaller than Cipro™ tablets.

It appears that Cipro™ tablets are made in accordance with the teaching of U.S. Pat. No. 5,286,754. More particularly, the formulation of Cipro™ tablets 100 mg, 250 mg, 500 mg and 750 mg appear to be precisely what is shown in examples 4, 2, 1, and 5 respectively of U.S. Pat. No. 5,286,754.

Cipro™ tablets are film-coated tablets, which means that they consist of core tablets made by compression on a tablet press, coated with a thin film coating.

The disclosure of U.S. Pat. No. 5,286,754 states that, when the active ingredient is ciprofloxacin hydrochloride monohydrate, the tablets may comprise by weight 60.0 to 90.0% active ingredient, 3.0 to 15.0% dry binder based on cellulose, 5.0 to 16.0% of a disintegrant based on starch, 1.0 to 7.0% of a disintegrant based on cellulose derivatives and/or crospovidone, 0.5 to 1.0% by weight of a glidant (flow improving agent), and 0.5 to 1.0% of a lubricant. However, the teachings do not appear to enable workable tablets with an active drug content of up to 90%. There is no example with an active drug content of above 77.73%; and, if the teachings enabled a tablet with a higher active drug content, then Cipro™ tablets presumably would have had a higher active drug content to enable the tablets to be smaller.

When one follows the teaching and, in particular, the examples of U.S. Pat. No. 5,286,754, the ability to achieve tablets with ciprofloxacin hydrochloride monohydrate content above 77.73% is limited by the following practicalities:

To avoid sticking and binding in the tabletting process, the formulation must include a lubricant. U.S. Pat. No. 5,286,754 states that: “Lubricants are, for example, talc, calcium stearate, magnesium stearate and solid polyethylene glycols. Magnesium stearate is preferred.” However, talc and solid polyethylene glycols give inadequate lubrication; and, when magnesium stearate or calcium stearate is used in an adequate quantity, they have an effect of slowing down the rate of dissolution of the tablet. This requires that disintegrants be added to the formulation to speed dissolution. It is presumably for this reason that the examples in U.S. Pat. No. 5,286,754 include both starch and crospovidone as disintegrants. Including these ingredients as disintegrants increases the size of the tablets. Moreover, the inclusion of starch in the tablets also reduces the hardness of the tablets, which also causes the need to include microcrystalline cellulose to increase tablet hardness. This increases the size of the tablets even further. It is for reasons that the active content in the examples of U.S. Pat. No. 5,286,754 and in Cipro™ tablets is only 77.73%.

In light of this prior art, an objective of the present invention is to enable tablets comprising ciprofloxacin hydrochloride monohydrate that have satisfactory hardness, and yet have an active drug content of above 77.73%.

### DESCRIPTION OF THE INVENTION

It has been found that, by minimizing the amount of starch used as disintegrant, and using a disintegrant selected from crospovidone, croscarmellose sodium, carmellose calcium, and sodium starch glycolate, it is possible to achieve tablets of adequate hardness with a ciprofloxacin hydrochloride content of over 77.73% by weight.

Tablets of the present invention comprise by weight over 80% ciprofloxacin hydrochloride monohydrate, less than 5% starch, and at least 0.5% of a disintegrant selected from crospovidone, croscarmellose sodium, carmellose calcium, and sodium starch glycolate. These compounds are more effective than starch as disintegrants, and hence, can enable adequate disintegration rate with use of a relatively small amount.

The tablets will preferably be free of starch.

The amount of disintegrant selected from crospovidone, croscarmellose sodium, carmellose calcium, and sodium starch glycolate by weight will preferably be from 0.6% to 5.0% and more preferably from 1.0% to 3.0%.

It has been found that the inclusion of a disintegrant selected from starch, croscarmellose sodium, carmellose calcium and sodium starch glycolate reduces the hardness of tablets comprising ciprofloxacin hydrochloride monohydrate, but the inclusion of crospovidone increases hardness. Thus the use of crospovidone as disintegrant in tablets of the present invention, further reduces the need for a dry binder based on cellulose to increase hardness. Hence, crospovidone is the most preferred disintegrant for tablets of the present invention; and use of crospovidone as the disintegrant is particularly effective in enabling tablets of good hardness that comprise by weight over 90% ciprofloxacin hydrochloride monohydrate.

The tablets will optionally comprise a relatively small amount of cellulose, as dry binder. The amount of cellulose, if any, will preferably be less then that 4.5% of the tablet by weight, and more preferably less than 3%. The tablets will most preferably be free of cellulose, in order to minimize tablet weight.

The tablets of the present invention will also contain a lubricant, which will preferably be a stearic acid salt, such as magnesium stearate, calcium stearate, or zinc stearate. The amount of lubricant by weight will preferably be from 0.5% to 3.0%, and more preferably from 1% to 2%.

The tablets will also optionally comprise a glidant. The glidant will preferably be colloidal silicon dioxide. The amount of glidant by weight will preferably be from 0.1% to 2.0%, and more preferably from 0.1% to 0.4%.

The total of all excipients in the tablets by weight will be under 20% of the tablet weight, so that the tablets will comprise over 80% ciprofloxacin hydrochloride monohydrate by weight. Preferably the total of all excipients by weight will be under 15%, so that the tablets will comprise over 85% ciprofloxacin hydrochloride monohydrate by weight. More preferably the total of all excipients by weight will be under 10%, so that the tablets will comprise over 90% ciprofloxacin hydrochloride monohydrate by weight. Most preferably, the total of all excipients by weight will be under 5%, so that the tablets will comprise over 95% ciprofloxacin hydrochloride by weight. Pharmaceutical tablets are conventionally made by either a wet-granulation process or dry-mix process.

In a wet-granulation process, ingredients are wetted with water or an organic solvent, which will optionally have a binder dissolved therein, and the wet mass is dried and milled into free-flowing granules. The granules are then mixed with other ingredients, and the mixture is then compressed into tablets.

A dry-mix process will be either a “direct-compression” process or a “dry-granulation” process. In a direct-compression process, the ingredients are mixed together in dry form, and the mixture is then directly compressed into tablets.

If, upon dry mixing, the mixture does not flow well enough for direct compression, a procedure known as “dry-granulation”, “compaction”, or “slugging” may be used. In this process, a mixture of ingredients will first be compacted into relatively large pieces known as “slugs” which are then milled into free flowing granules.

These granules are then compressed into the final tablets.

A dry-mix process is generally more economical and preferred to a wet-granulation process. However, a dry-mix process cannot always be achieved.

It has been found that tablets of the present invention can be made by a dry-mix process, and in particular a dry-granulation process. Hence, the tablets of the present invention will preferably be made by a dry-mix process, which will preferably be a dry-granulation process.

Because ciprofloxacin hydrochloride has an unpleasant taste, the tablets will preferably be film-coated to cover the taste. For a film-coated tablet, it will be understood that the percentages given herein for ingredients are relative to the weight of the core tablet, excluding the film-coating.

The invention will be better understood from the following example, which is intended to be illustrative of the invention and not limiting.

### EXAMPLE 1

Ingredients were mixed in the following proportions, without addition of any solvent.

| | |
|---|---|
| Ciprofloxacin HCl monohydrate | 873.0 |
| Crospovidone | 18.0 |
| Magnesium stearate | 13.5 |
| Colloidal silicon dioxide | 1.5 |
| | 906.0 |

The mixture was compressed into slugs on a tablet press. The slugs were then milled into granules. The granules were then remixed, and the mixture was recompressed into tablets of weight 906 mg per tablet. Each tablet thus contained 873 mg of ciprofloxacin hydrochloride monohydrate, equivalent to 750 mg of ciprofloxacin.

The tablets had satisfactory hardness, and a disintegration time of only a few minutes in water.

The tablets of this example have an excipient content of under 5% by weight, and thus comprise over 95% ciprofloxacin hydrochloride monohydrate by weight.

1. A tablet which comprises by weight more than 80% ciprofloxacin hydrochloride monohydrate, less than 5% starch, and at least 0.5% of a disintegrant selected from the group consisting of crospovidone, croscarmellose sodium, carmellose calcium, and sodium starch glycolate.

2. A tablet of claim 1 which comprises more than 85% ciprofloxacin hydrochloride monohydrate by weight.

3. A tablet of claim 2 which comprises more than 90% ciprofloxacin hydrochloride monohydrate by weight.

4. A tablet of claim 3 which comprises more than 95% ciprofloxacin hydrochloride monohydrate by weight.

5. A tablet of any of claims 1 to 4 which is free of starch.

6. A tablet of any of claims 1 to 5 wherein the amount of said disintegrant is from 0.6% to 5.0% of the tablet by weight.

7. A tablet of claim 6 wherein the amount of said disintegrant is from 1.0% to 3.0% of the tablet by weight.

8. A tablet of any of claims 1 to 7 wherein said disintegrant is crospovidone.

9. A tablet of any of claims 1 to 8 which further comprises cellulose, and wherein the amount of cellulose is less than 4.5% of the tablet by weight.

10. A tablet of claim 9 wherein the amount of said cellulose is less than 3% of the tablet by weight.

11. A tablet of any of claims 1 to 8 which is free of cellulose.

12. A tablet of any of claims 1 to 11 which further comprises a lubricant.

13. A tablet of claim 12, wherein the lubricant is a stearic acid salt.

14. A tablet of claim 12 or 13, wherein the amount of lubricant is from 0.5% to 3.0% of the tablet by weight.

15. A tablet of any of claims 1 to 14 which further comprises colloidal silicon dioxide.

16. A tablet which comprises by weight more than 90% ciprofloxacin hydrochloride monohydrate, less than 5% starch, and at least 0.5% crospovidone.

17. A tablet of claim 16 which comprises more than 95% ciprofloxacin hydrochloride monohydrate.

18. A tablet of claim 16 to 17 which is free of starch.

19. A tablet of any of claims 16 to 18 wherein the amount of crospovidone is from 0.6% to 5.0% of the tablet by weight.

20. A tablet of claim 19 wherein the amount of crospovidone is from 1.0% to 3.0% of the tablet by weight.

21. A tablet of any of claims 16 to 20 which further comprises cellulose, and wherein the amount of cellulose is less than 4.5% of the tablet by weight.

22. A tablet of any of claims 16 to 20 which is free of cellulose.

23. A tablet of any of claims 16 to 22 which further comprises a lubricant.

24. A tablet of claim 23 wherein the lubricant is a stearic acid salt.

25. A tablet of claim 23 or 24 wherein the amount of lubricant is from 0.5% to 3.0% of the tablet by weight.

26. A tablet of any of claims 16 to 25 which further comprises colloidal silicon dioxide.

27. A tablet of any of claims 1 to 26, when made by a dry-mix process.

28. A tablet of any of claims 1 to 26, when made by a dry-granulation process.
